(19) United States
(12) Patent Application Publication
(10) Pub. No.: US 2004/0082827 A1
(43) Pub. Date: Apr. 29, 2004

(54) N-butane and ethylene conversion process

(76) Inventor: Bruce B. Randolph, Bartlesville, OK (US)

Correspondence Address: RICHMOND, HITCHCOCK,, FISH & DOLLAR, P.O. Box 2443, Bartlesville, OK, 74005, US

(21) Appl. No.: 10/280,422

(22) Filed: Oct. 25, 2002

(51) Int. Cl. C07C 2/56; C07C 2/58

(52) U.S. Cl. 585/721; 585/723; 585/727

(57) ABSTRACT

A process for converting n-butane and ethylene to a product by contact with a catalyst containing a Group VIII metal, a halogen, and a support, in the presence of a Lewis Acid, and optionally in the presence of hydrogen, is disclosed.

### BACKGROUND OF THE INVENTION

The present invention relates to the field of hydrocarbon upgrading processes. In another aspect, the invention relates to the conversion of n-butane and ethylene to C₅+hydrocarbons.

Petroleum refiners produce large quantities of n-butane. Some of this n-butane can be blended into gasoline, but Reid Vapor Pressure limitations limit the amount which can be used. Some n-butane can be isomerized to isobutane for use in alkylation or dehydrogenation. However, excess n-butane is not uncommon in a refinery. Similarly, ethylene is produced by refineries and is often stranded in fuel gas wherein the low purity and the presence of numerous contaminants makes traditional polymerization processes unacceptable. Thus, significant quantities of n-butane and ethylene become orphan streams in the refinery and are often simply burned as fuel gas (ethylene) or sold at discounted prices as LPG (n-butane). Therefore, development of a process for converting n-butane and ethylene to useful products, such as gasoline range hydrocarbons, would be a significant contribution to the art and to the economy.

### SUMMARY OF THE INVENTION

It is an object of the present invention to provide a process for converting n-butane and ethylene to a product.

It is yet another object of the present invention to provide a process for converting n-butane and ethylene to C₅+hydrocarbons wherein gallium chloride is present in the feed.

In accordance with the present invention, a process for converting n-butane and ethylene to a product has been found and comprises:

contacting a feed stream comprising n-butane, ethylene and a Lewis Acid in a reaction zone, under reaction conditions, with a catalyst comprising a halogen, a Group VIII metal and a support, to thereby form a product.

Other objects and advantages will become apparent from the detailed description and the appended claims.

### DETAILED DESCRIPTION OF THE INVENTION

The catalyst useful in the present invention comprises, consists of, or consists essentially of a halogen, a Group VIII metal and a support. The halogen is selected from the group consisting of fluorine, chlorine, bromine and combinations thereof. The most preferred halogen is chlorine.

The Group VIII metal is selected from the group consisting of platinum, palladium, nickel, cobalt, rhodium, iridium, osmium, ruthenium, iron, and combinations of any two or more thereof. Preferably, the Group VIII metal is platinum or palladium, and most preferably platinum.

The support is selected from the group consisting of alumina, silica-alumina, activated carbon, and combinations of any two or more thereof. The support is preferably alumina.

The halogen is present in the catalyst in an amount in the range of from about 1 wt. % to about 7 wt. %, preferably in the range of from about 2 wt. % to about 6 wt. %, and most preferably in the range of from about 3 wt. % to about 5 wt. %, based on the total weight of the catalyst.

The Group VIII metal is present in the catalyst in an amount in the range of from about 0.1 wt. % to about 0.5 wt. %, preferably in the range of from about 0.15 wt. % to about 0.4 wt. %, and most preferably in the range of from about 0.2 wt. % to about 0.3 wt. %, based on the total weight of the catalyst.

This invention involves a process for contacting a feed stream comprising, consisting of, or consisting essentially of n-butane, ethylene and a Lewis Acid in a reaction zone, under reaction conditions, with a catalyst as hereinabove described, to thereby form a product. The Lewis Acid is preferably a Group III A metal halide. The Lewis Acid is more preferably gallium chloride. The reaction zone can also contain hydrogen. In addition, the feed stream is preferably substantially perchloroethylene free, more particularly, the feed stream contains less than 1 ppmw perchloroethylene based on the total weight of the feed stream.

The reaction conditions include a temperature in the range of from about 200° F. to about 300° F., preferably from about 220° F. to about 280° F.

The Lewis Acid is present in the feed stream in an amount in the range of from about 100 ppmw to about 200 ppmw, preferably in an amount in the range of from about 125 ppmw to about 175 ppmw, based on the total weight of said feed stream.

n-Butane is present in the feed stream in an amount in the range of from about 50 wt. % to about 99 wt. %, preferably in an amount in the range of from about 90 wt. % to about 95 wt. %, based on the total weight of said feed stream.

Ethylene is present in the feed stream in an amount in the range of from about 1 wt. % to about 50 wt. %, preferably in an amount in the range of from about 5 wt. % to about 10 wt. %, based on the total weight of said feed stream.

The following example is presented to further illustrate the invention and is not be construed as unduly limiting its scope.

### EXAMPLE

In this example, lab-scale tests are described to illustrate the process of this invention.

Run I

A stainless-steel reactor (having an inner diameter of about 0.75 inch and a height of about 28 inches) was filled with a layer (about 13.5 inches high) of Alundum® (inert alumina particles having a surface area of 1 m²/g or less), a layer (about 6 inches high) of a Pt/alumina isomerization catalyst containing about 0.2 weight-% Pt, about 45 weight-% Al, about 3.9 weight-% Cl, and ≦0.2 weight-% Ti or Zr, and the remainder being essentially chemically bound oxygen and hydrogen; surface area: 195 m²/g) and a top layer (about 8 inches high) of Alundum®.

The reactor contents were heated to about 239.8° F. in the presence of hydrogen, and a liquid feed (containing about 94 weight percent normal butane, about 0.34 weight percent iso-butane and about 5.48 weight percent ethylene), was introduced into the reactor at a liquid-volume hourly space velocity of about 4 hours⁻¹. The liquid feed also contained about 0.09 weight percent propane. The reaction pressure was about 300 pounds per square inch gauge (psig).

Gallium chloride was added to the liquid feed in an amount so as to maintain a concentration of about 167 ppmw of gallium chloride in the liquid feed (i.e., about 167 parts by weight gallium chloride per million parts by weight of at least one feed hydrocarbon) along with added hydrogen. The hydrogen was introduced in an amount so as to provide a hydrogen-to-hydrocarbon (H₂:HC) molar ratio of about 0.016:1.

The obtained product (primarily containing isoparaffins, olefins and cycloparaffins) was analyzed by means of a gas chromatograph. Results from Run I are summarized in the Table.

Run II

Run II was conducted in the same way as Run I, with the following exceptions: 1) the reactor contents were heated to about 244.0° F. and, 2) the hydrogen was introduced in an amount so as to provide a molar H₂:HC ratio of about 0.008:1. Results from Run II are summarized in the Table.

TABLE

| Products (wt. %) | Run I | Run II |
|---|---|---|
| C₁-C₄ | 89.066 | 86.963 |
| C₅+ | 10.934 | 13.037 |
| C₅ + Product Breakdown | | |
| Paraffins | 2.31 | 1.25 |
| Isoparaffins | 35.72 | 40.78 |
| Olefins | 24.19 | 13.33 |
| Naphthenes | 36.17 | 38.71 |
| Aromatics | 0.70 | 1.96 |
| C₁₃+ | 0.00 | 0.09 |
| Unknowns | 0.91 | 3.88 |
| Total | 100.00 | 100.00 |
| C6 Isoparaffins, wt. % | 13.3 | 6.16 |
| *C6 Olefins, wt. % | 6.93 | 6.27 |
| C7 Olefins, wt. % | 17.26 | 6.81 |
| Napthenes Breakdown, wt. % | | |
| C6 | 12.69 | 6.76 |
| C7 | 13.51 | 15.09 |
| C8 | 7.54 | 8.75 |
| C9 | 2.03 | 5.98 |
| C10+ | 0.40 | 2.14 |
| Aromatics Breakdown, wt. % | | |
| C8 | 0.05 | 0.16 |
| C9 | 0.56 | 0.75 |
| C10+ | 0.09 | 1.05 |

*only C6 olefin observed was cyclohexene

The data from Runs I and II clearly demonstrate the ability of the inventive process to convert n-butane and ethylene to gasoline range hydrocarbons.

Reasonable variations, modifications and adaptations for various conditions and reactants can be made within the scope of the disclosure and the appended claims without departing from the scope of this invention.

That which is claimed is:

1. A process comprising:
contacting a feed stream comprising n-butane, ethylene and a Lewis Acid in a reaction zone, under reaction conditions, with a catalyst comprising a halogen, a Group VIII metal and a support, to thereby form a product.

2. A process in accordance with claim 1 wherein said Lewis Acid is a Group III A metal chloride.

3. A process in accordance with claim 1 wherein said Lewis Acid is gallium chloride.

4. A process in accordance with claim 1 wherein said reaction conditions include a temperature in the range of from about 200° F. to about 300° F.

5. A process in accordance with claim 1 wherein said reaction conditions include a temperature in the range of from about 220° F. to about 280° F.

6. A process in accordance with claim 1 wherein said reaction zone contains hydrogen.

7. A process in accordance with claim 1 wherein said Lewis Acid is present in said feed stream in an amount in the range of from about 100 ppmw to about 200 ppmw, based on the total weight of said feed stream.

8. A process in accordance with claim 1 wherein said Lewis Acid is present in said feed stream in an amount in the range of from about 125 ppmw to about 175 ppmw, based on the total weight of said feed stream.

9. A process in accordance with claim 1 wherein said n-butane is present in said feed stream in an amount in the range of from about 50 wt. % to about 99 wt. %, based on the total weight of said feed stream.

10. A process in accordance with claim 1 wherein said n-butane is present in said feed stream in an amount in the range of from about 90 wt. % to about 95 wt. %, based on the total weight of said feed stream.

11. A process in accordance with claim 1 wherein said ethylene is present in said feed stream in an amount in the range of from about 1 wt. % to about 50 wt. %, based on the total weight of said feed stream.

12. A process in accordance with claim 1 wherein said ethylene is present in said feed stream in an amount in the range of from about 5 wt. % to about 10 wt. %, based on the total weight of said feed stream.

13. A process in accordance with claim 1 wherein said halogen is selected from the group consisting of fluorine, chlorine, bromine and combinations thereof.

14. A process in accordance with claim 1 wherein said halogen is chlorine.

15. A process in accordance with claim 1 wherein said Group VIII metal is selected from the group consisting of platinum, palladium, nickel, cobalt, rhodium, iridium, osmium, ruthenium, iron, and combinations of any two or more thereof.

16. A process in accordance with claim 1 wherein said Group VIII metal is platinum.

17. A process in accordance with claim 1 wherein said Group VIII metal is palladium.

18. A process in accordance with claim 1 wherein said support is selected from the group consisting of alumina, silica-alumina, activated carbon, and combinations of any two or more thereof.

19. A process in accordance with claim 1 wherein said support is alumina.

20. A process in accordance with claim 1 wherein said halogen is present in said catalyst in an amount in the range of from about 1 wt. % to about 7 wt. %, based on the total weight of said catalyst.

21. A process in accordance with claim 1 wherein said halogen is present in said catalyst in an amount in the range of from about 3 wt. % to about 5 wt. %, based on the total weight of said catalyst.

22. A process in accordance with claim 1 wherein said Group VIII metal is present in said catalyst in an amount in the range of from about 0.1 wt. % to about 0.5 wt. %, based on the total weight of said catalyst.

23. A process in accordance with claim 1 wherein said Group VIII metal is present in said catalyst in an amount in the range of from about 0.2 wt. % to about 0.3 wt. %, based on the total weight of said catalyst.
